(19) United States
(12) Patent Application Publication
(10) Pub. No.: US 2010/0286558 A1
(43) Pub. Date: Nov. 11, 2010

(54) FLUID COLLECTION/INJECTION DEVICE HAVING QUICK RELEASE/REMOVABLE DOUBLE-ENDED NEEDLE AND SAFETY SYSTEM

(75) Inventor: Steven SCHRAGA, Surfside, FL (US)

Correspondence Address: GREENBLUM & BERNSTEIN, P.L.C., 1950 ROLAND CLARKE PLACE, RESTON, VA, 20191, US

(73) Assignee: STAT MEDICAL DEVICES, INC., North Miami, FL (US)

(21) Appl. No.: 12/755,917

(22) Filed: Apr. 7, 2010

Related U.S. Application Data
(60) Provisional application No. 61/167,718, filed on Apr. 8, 2009.

(51) Int. Cl. A61M 5/34; A61M 5/50; A61B 5/15

(52) U.S. Cl. 600/576; 604/240; 604/241; 604/110

(57) ABSTRACT

Fluid collection/injection device includes a body having a front end, a back end, and a main hollow section arranged between the front and back ends. A needle hub securing section is arranged on the front end and is structured and arranged to receive therein a needle member. The needle hub securing section includes a fixed part and a movable part. The fixed part is integrally formed with the front end and the movable part is arranged on a member that has one end which is one of fixed to a portion of the main hollow section, connected to a portion of the main hollow section via a living hinge, removably connected to a portion of the main hollow section, and integrally formed with the main hollow section.

### CROSS-REFERENCE TO RELATED APPLICATIONS

The instant application is a U.S. non-provisional Application based on U.S. provisional application No. 61/167,718, filed Apr. 8, 2009, the disclosure of which is hereby expressly incorporated by reference hereto in its entirety.

### BACKGROUND OF THE INVENTION

1. Field of the Invention

This invention relates generally to devices used to collect fluid samples from and/or inject fluids into patients. More specifically, this invention relates to a device which utilizes a holder having a double-ended needle that can be released or removed from the holder in a more safe and easy manner. The device can be a single-use device. The invention also relates to a method of collecting a fluid sample with the device as well as a method of making the device. The invention also relates to a blood sample collection device that is less costly to produce and/or easier to manufacture.

2. Discussion of Background Information

Prevention of needle sticks is of paramount concern in the healthcare industry because of serious and deadly risk factors associated with AIDS and other serious communicable diseases. Typical blood collection devices utilize a needle inserted into a patient's vein so as to draw blood through the needle into an associated separate collection reservoir. Accidental needle sticks from previously used needles can occur during the fluid withdrawing process and subsequent handling and disposal operation. Until such used medical devices are destroyed, they remain a risk to those handling them.

Devices used for blood sampling are well know and include a collection device sold under the trademark Vacutainer® by Becton Dickinson Corporation. This device has a tubular syringe-like body with a needle in the front end, part of which extends back into a tubular syringe-like shell. Part of the needle extends externally for punching the skin. An evacuated collection tube with a rubber stopper is placed into the open back of the syringe-like shell with the rubber stopper against the internal end of the needle. After the skin is punctured, the collection tube is pushed forward to cause the needle to enter the evacuated tube. Vacuum helps draw blood into the collecting tube. When a sufficient sample has been obtained, the collecting tube and the stopper are simply withdrawn from the tubular shell and sent to the laboratory. This particular device has a permanently extended needle and an opening in the back for the collection tube which remains open after the collection tube is removed, leaving small quantities of blood and an internally exposed needle.

Medical devices which are used for collecting fluid samples from patients which have quick release needle systems are also known. Such devices include: U.S. Pat. No. 5,797,490 to FUJI et al; U.S. Pat. No. 5,755,673 to KINSEY; U.S. Pat. No. 4,822,343 to BEISER; U.S. Pat. No. 4,984,580 WANAMAKER; U.S. Pat. No. Re. 38,964 to SHILLINGTON; U.S. Pat. No. 5,616,136 to SHILLINGTON et al.; U.S. Pat. No. 5,637,101 to SHILLINGTON; U.S. Pat. No. 5,117,837 to WANAMAKER et al.; U.S. Pat. No. 4,907,600 to SPENCER; U.S. Pat. No. 4,993,426 to SPENCER; U.S. Pat. No. 4,904,244 to HARSH et al.; U.S. Pat. No. 4,490,142 to SILVERN. The disclosures of each of these documents is expressly incorporated by reference herein in their entireties.

Embodiments of the invention aim to improve devices of the type described above by making a fluid collection holder which is easier to make and use and/or cheaper to make so that its use can be more widespread. The device is also believed to be as safe or safer to use and/or dispose-of than the above-noted devices.

### SUMMARY OF THE INVENTION

According to one non-limiting aspect of the invention there is provided a fluid collection/injection device comprising a body having a front end, a back end, and a main hollow section arranged between the front and back ends, a needle hub securing section arranged on the front end and being structured and arranged to receive therein a needle member, and the needle hub securing section comprises a fixed part and a movable part. The fixed part is integrally formed with the front end and the movable part is arranged on a member that has one end which is one of fixed to a portion of the main hollow section, connected to a portion of the main hollow section via a living hinge, removably connected to a portion of the main hollow section, and integrally formed with the main hollow section.

The body may be one of generally cylindrical and generally tubular. The fixed part and the movable part may form a generally cylindrical section which the movable part is in an initial position. The fixed part and the movable part may each generally comprise one-half of an internal locking thread structured and arranged to engage with an external thread of the needle member. The member may have one end which is fixed to the portion of the main hollow section. The member may have one end which is removably connected to the portion of the main hollow section. The member may have one end which is integrally formed with the main hollow section. The member may have one end which is connected to the portion of the main hollow section via the living hinge.

The body and the needle hub securing section may comprise a one-piece member. The device may be a one-piece member. The device may further comprise guide projections arranged on the front end of the body. The member may comprise a projection for facilitating removal of the needle member. The member may comprise an opening sized to receive therein the fixed part. The member may comprise a finger engaging section which is arranged on a side of the fixed part opposite the movable part. The member may comprise a finger engaging section which is movable towards the body when it experiences a force and which moves back to an initial position away from the body when the force is removed. The member may comprise a finger engaging section which is movable towards the body when it experiences a force and which automatically moves back to an initial position away from the body when the force is removed. The front end may comprise a through opening sized to receive an inner needle end of the needle member. The rear end may comprise a flange. The rear end may comprise a rotatable and/or movably mounted flange.

The device may further comprise a mechanism for locking the movable part in a position which prevents the removable needle member from being reinstalled or locked to the body. The device may further comprise a mechanism for preventing re-use of the device. The device may further comprise a mechanism for rendering the device single-use. The device may further comprise a biasing mechanism biasing the movable part towards the fixed part.

The invention also provides for a method of taking a fluid sample using the device described above, wherein the method comprises installing the needle member onto the device, inserting a receptacle into the device, and removing the receptacle from the device.

The invention also provides for a method of taking a fluid sample using the device described above, wherein the method comprises inserting a receptacle into the device and removing the receptacle from the device.

The invention also provides for a method of taking a fluid sample using the device described above, wherein the method comprises inserting a receptacle into the device, removing the receptacle from the device, and preventing re-use of the device.

The invention also provides for a sampling or injecting device comprising a generally cylindrical body having a front end, a back end, and a main hollow section arranged between the front and back ends, a needle hub securing section arranged on the front end and being structured and arranged to receive therein a removable needle member, and the needle hub securing section comprises a fixed part and a movable part which together function to secure the removable needle member to the body. The fixed part is integrally formed with the front end and the movable part is arranged on a member that has one end which is one of fixed to a portion of the main hollow section, connected to a portion of the main hollow section via a living hinge, removably connected to a portion of the main hollow section, and integrally formed with the main hollow section.

The invention also provides for a blood sampling device comprising a body having a front end, a back end, and a main hollow section arranged between the front and back ends, a needle hub securing section arranged on the front end and being structured and arranged to receive therein a needle member, and the needle hub securing section comprises a fixed part and a movable part which together function to secure the removable needle member to the body. The fixed part is integrally formed with the front end and the movable part is arranged on a member that has one end which is one of fixed to a portion of the main hollow section, connected to a portion of the main hollow section via a living hinge, removably connected to a portion of the main hollow section, and integrally formed with the main hollow section.

The invention also provides for a method of taking a fluid sample using a fluid sampling device comprising a releasable securing mechanism having one end coupled to a side wall of a body and another end which engages with a needle member and is movably retained to a front end of the body. The method comprises inserting a receptacle into the body of the device and removing the receptacle from the device. The method may further comprise preventing re-use of the device.

Other exemplary embodiments and advantages of the present invention may be ascertained by reviewing the present disclosure and the accompanying drawing.

### BRIEF DESCRIPTION OF THE DRAWINGS

The present invention is further described in the detailed description which follows, in reference to the noted plurality of drawings by way of non-limiting examples of exemplary embodiments of the present invention, in which like reference numerals represent similar parts throughout the several views of the drawings, and wherein:

FIG. 1 shows a side cross-section view of a first non-limiting embodiment of a fluid sampling device according to the invention. The needle member is shown in an installed condition and is not shown in cross-section;

FIGS. 2 and 3 show side views of a needle member that can be used in the fluid sampling device of FIG. 1. In FIG. 2, the needle member is shown in an original position with its rear needle covered. In FIG. 3, the needle member is shown in a use position with its rear needle cover in a retracted position exposing the tip of the rear needle. This occurs when the rear needle is caused to puncture a sampling container after it is inserted into the sampling device shown in FIG. 1;

FIG. 4 shows a side view of the fluid sampling device of FIG. 1, but rotated about 90 degrees and with the needle member removed. FIG. 4 shows the releasable securing mechanism in a pre-bent configuration;

FIG. 5 shows a side view of the fluid sampling device of FIG. 4, but rotated about 90 degrees, and showing the releasable securing mechanism deflected outwardly by a small amount;

FIG. 6 shows a side view of the fluid sampling device of FIG. 1, but with the needle member removed. In FIG. 6, the free end of the releasable securing mechanism has been bent about 90 degrees from the position shown in FIG. 5 to that shown in FIG. 6;

FIG. 7 shows a front end view of the fluid sampling device of FIG. 6, except that the rear flange of the fluid sampling device is not illustrated. As was the case in FIG. 6, the releasable securing mechanism is biased to the closed position;

FIG. 8 shows a cross-section view of the fluid sampling device of FIG. 8;

FIG. 9 shows a front end view of the fluid sampling device of FIG. 8. The rear flange of the fluid sampling device is not illustrated;

FIG. 10 shows a side view of the fluid sampling device of FIG. 6, but with the releasable securing mechanism being disposed in the open position. The open position occurs when a force is applied to a portion of the releasable securing mechanism sufficient to deflect it from an original position. In this open position, the needle member can be removed and/or installed onto/into the fluid sampling device;

FIG. 11 shows a front end view of the fluid sampling device of FIG. 10. The rear flange of the fluid sampling device is not illustrated.;

FIG. 12 shows a cross-section view of the fluid sampling device of FIG. 10;

FIG. 13 shows a front end view of the fluid sampling device of FIG. 12. The rear flange of the fluid sampling device is not illustrated;

FIG. 14 shows a side view of the fluid sampling device of FIG. 4, except that a free end of the releasable securing mechanism is removed to better illustrate a front end of the fluid sampling device;

FIG. 15 shows an enlarged portion of a front end of FIG. 8, except that a free end of the releasable securing mechanism is removed to better illustrate a front end of the fluid sampling device;

FIG. 16 shows a front end view of the fluid sampling device of FIG. 15;

FIG. 17 shows an enlarged portion of FIG. 8, except that front and rear ends of the body of the fluid sampling device are removed to better illustrate the releasable securing mechanism;

FIG. 18 shows a side cross-section view of a second non-limiting embodiment of a fluid sampling device according to the invention. This embodiment is similar to that of FIG. 1 and utilizes a different needle member. The needle member is shown in an installed condition and is not shown in cross-section;

FIG. 19 shows a side view of the needle member used in the fluid sampling device of FIG. 18. In FIG. 19, the needle member is shown in an original position with its rear needle covered and with a biasing member in an original non-retracted state. The retracted state of the biasing member is shown in FIG. 18;

FIG. 20 shows a front end view of another embodiment of the fluid sampling device, except that the rear flange of the fluid sampling device is not illustrated. This device is similar to that shown in FIG. 7, but rotated about 90 degrees and additionally utilizing a spring to assist in biasing the releasable securing mechanism towards the closed position;

FIG. 21 shows the front end view of FIG. 20, but with the releasable securing mechanism being disposed in the open position;

FIG. 22 shows a front end view of another embodiment of the fluid sampling device, except that the rear flange of the fluid sampling device is not illustrated. This device is similar to that shown in FIG. 21, but utilizes an open slot configuration as well as side slots to make it easier to fully install a free end of the releasable securing mechanism on the fluid sampling device. The releasable securing mechanism is shown in the open position which also facilitates installation;

FIG. 23 shows the front end view of FIG. 22, but with the releasable securing mechanism being disposed in the closed position;

FIG. 24 shows an enlarged partial side view of a front portion of the fluid sampling device shown in FIG. 23, except that the releasable securing mechanism is removed to better illustrate a front end of the fluid sampling device;

FIG. 25 shows a front end view of the fluid sampling device of FIG. 24;

FIG. 26 shows a side view of another embodiment of a fluid sampling device. The device is similar to that shown in FIG. 5, except that the releasable securing mechanism has a rear end that is non-integrally formed with the body of the fluid sampling device and/or is removable secured thereto;

FIG. 27 shows a side view of the fluid sampling device of FIG. 26, but rotated 90 degrees;

FIG. 28 shows a side view of another embodiment of a fluid sampling device. The device is similar to that shown in FIG. 27, except that the releasable securing mechanism has a rear end that is received in a recess formed in the body of the fluid sampling device;

FIG. 29 shows a side view of the fluid sampling device of FIG. 28, but with the releasable securing mechanism removed to illustrate the recess formed in the body;

FIG. 30 shows a side view of the releasable securing mechanism used in the embodiment shown in FIG. 28;

FIG. 31 shows a rear end view of only the rear end of the releasable securing mechanism of FIG. 30;

FIG. 32 shows an enlarged partial side view of a front portion of the fluid sampling device according to another embodiment of the invention. The releasable securing mechanism is shown in a closed position;

FIG. 33 shows a front end view of the fluid sampling device of FIG. 32;

FIG. 34 shows the fluid sampling device of FIG. 32, except that the releasable securing mechanism is shown in an open position;

FIG. 35 shows a front end view of the fluid sampling device of FIG. 34;

FIG. 36 shows a side view of another embodiment of a fluid sampling device. The device is similar to that shown in FIG. 27, except that the releasable securing mechanism has a rear end that is non-integrally formed with the body of the fluid sampling device and is non-removably secured thereto;

FIG. 37 shows a side view of the fluid sampling device of FIG. 36, but rotated 90 degrees. The rear end of the releasable securing mechanism is shown in cross-section and illustrates how it connected to the body via securing projections and openings;

FIG. 38 shows an enlarged partial view of the rear end shown in FIG. 37 and illustrates one securing projection and opening;

FIG. 39 shows a rear end view of another non-limiting embodiment of a fluid sampling device according to the invention. This embodiment is similar to FIG. 1, except that the rear flange of the body of the fluid sampling device is a separately formed member and/or that can rotate relative to the body thereof;

FIG. 40 shows an enlarged side cross-section view of the rear end of the body of the fluid sampling device shown in FIG. 40;

FIG. 41 shows a side cross-section view of another non-limiting embodiment of a fluid sampling device according to the invention. The releasable securing mechanism is shown in an open position and is locked in this position to prevent re-use of the fluid sampling device;

FIG. 42 shows an enlarged view of a locking portion of the fluid sampling device shown in FIG. 41;

FIG. 43 shows a side view of another non-limiting embodiment of a fluid sampling device according to the invention. The releasable securing mechanism is shown in an open position;

FIG. 44 shows a side view of the fluid sampling device of FIG. 43, but with the releasable securing mechanism being shown in a closed position;

FIG. 45 shows an enlarged partial side view of a front end of another non-limiting embodiment of a fluid sampling device according to the invention. The releasable securing mechanism is shown in a closed position and is biased to the closed position by the illustrated elastic ring;

FIG. 46 shows a side view of another embodiment of a fluid sampling device. The device is similar to that shown in FIG. 36, except that the releasable securing mechanism has an integrally formed biasing member which functions like a spring; and

FIG. 47 shows a side view of the fluid sampling device of FIG. 46, but rotated 90 degrees.

### DETAILED DESCRIPTION OF THE INVENTION

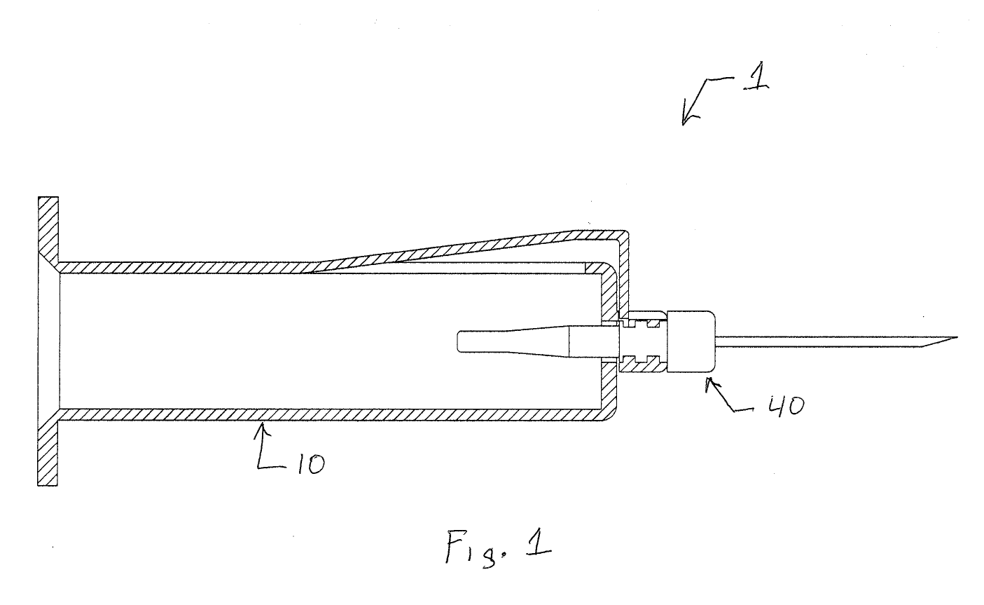

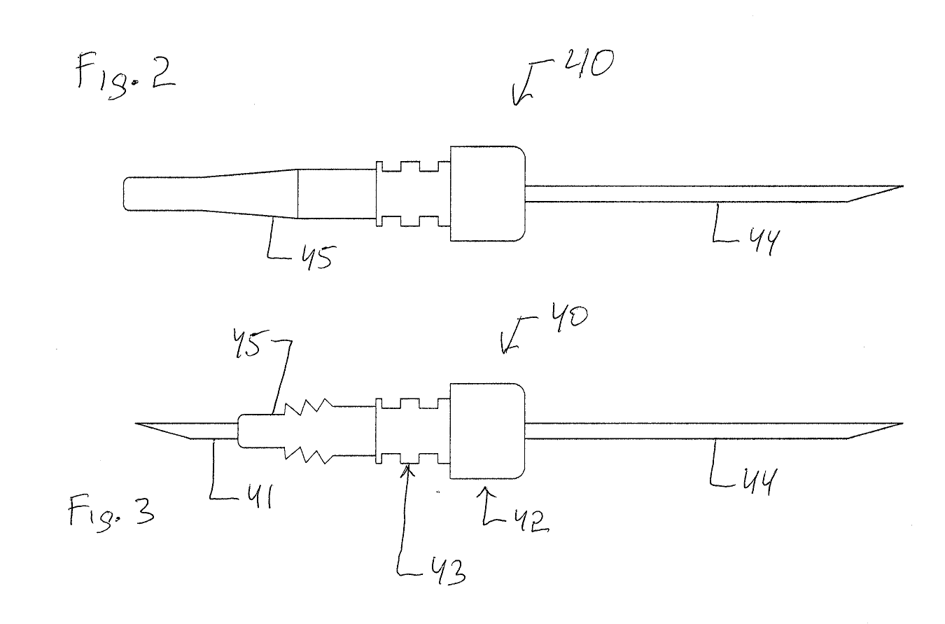

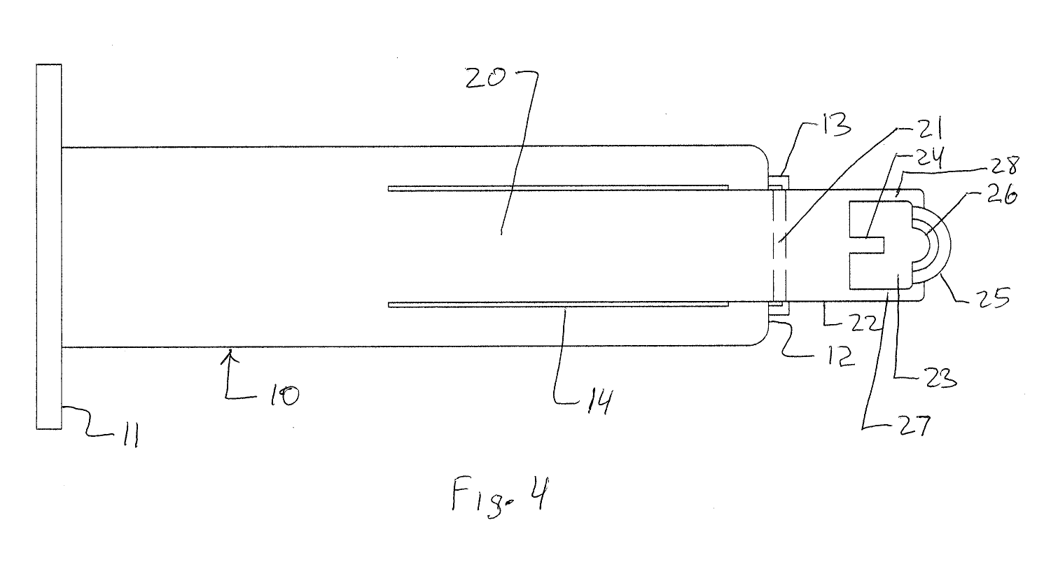

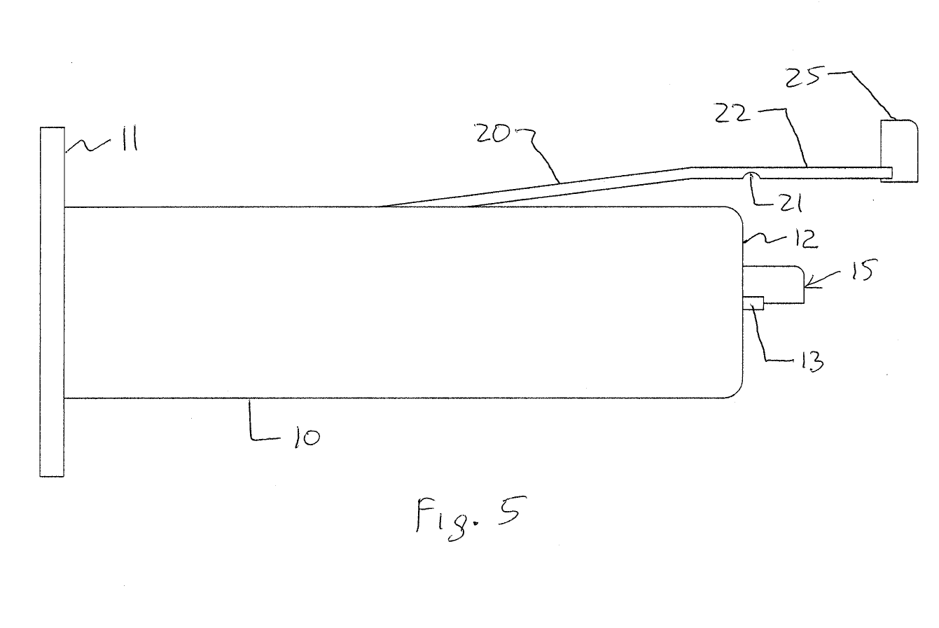

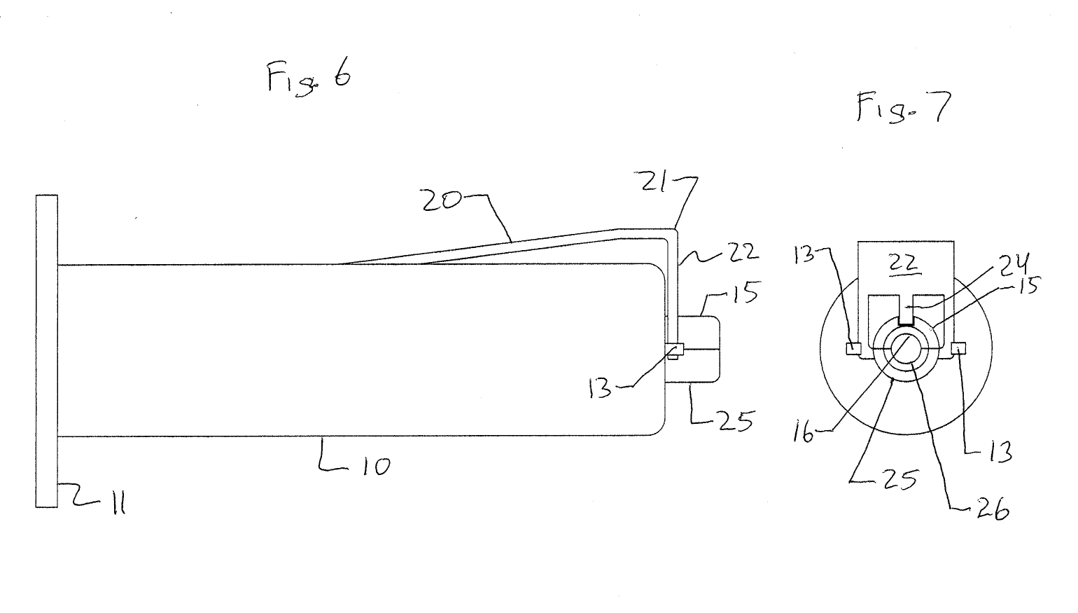

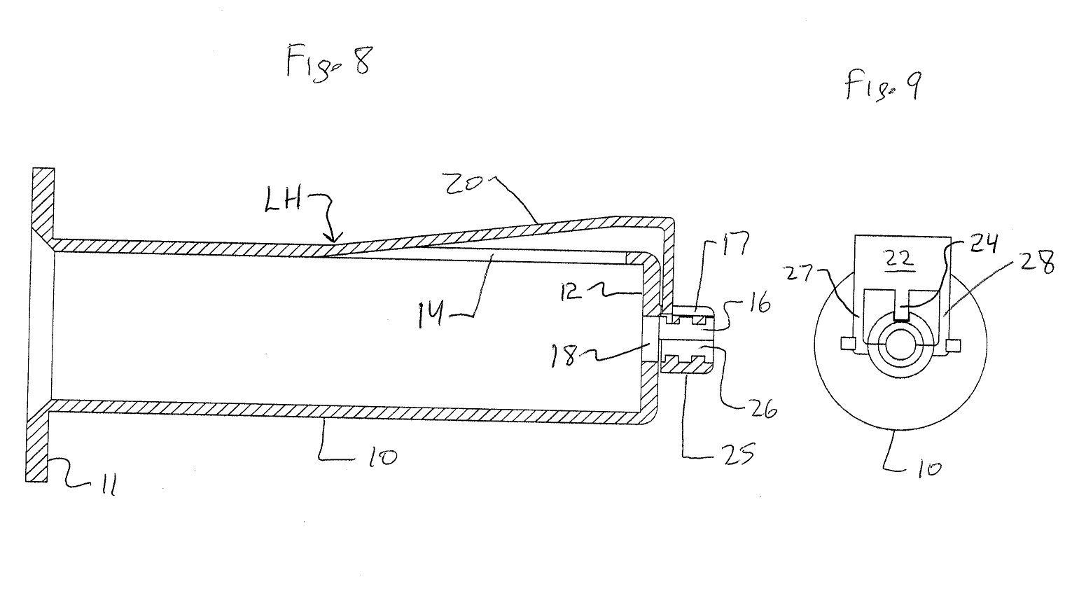

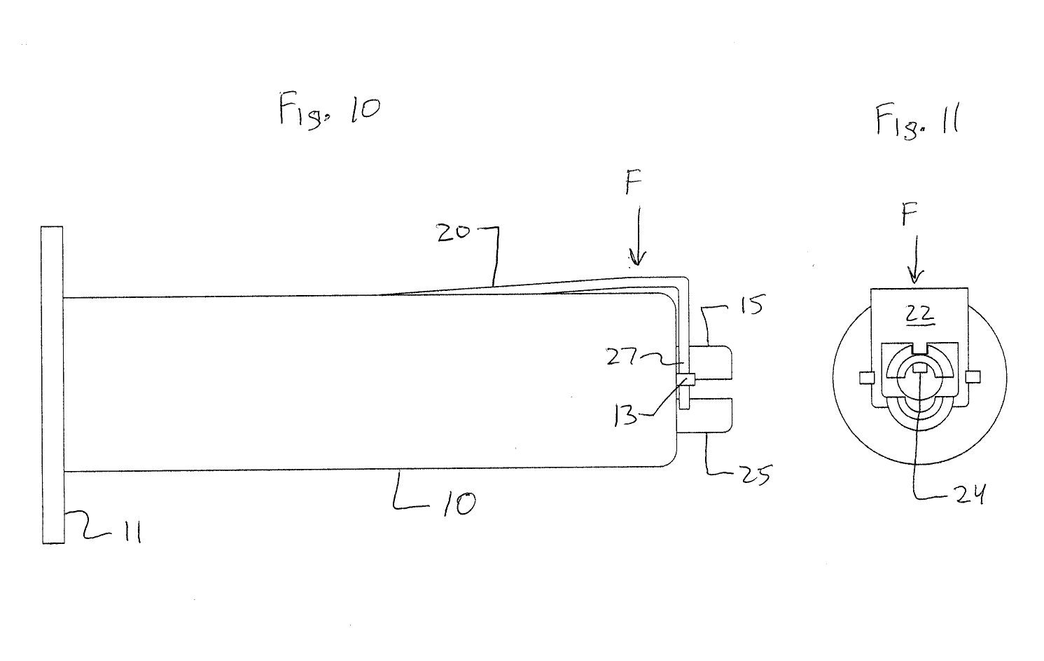

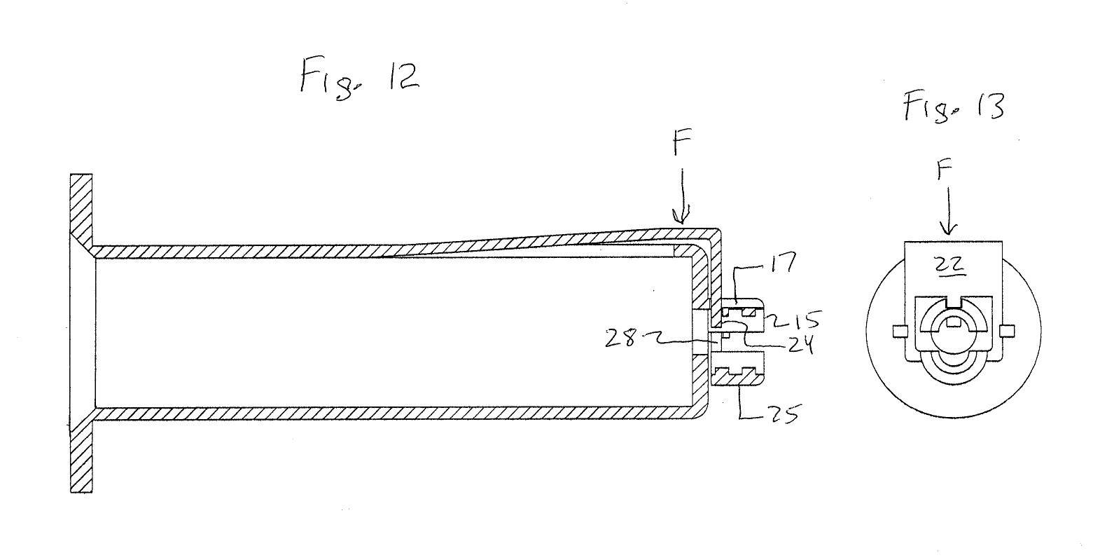

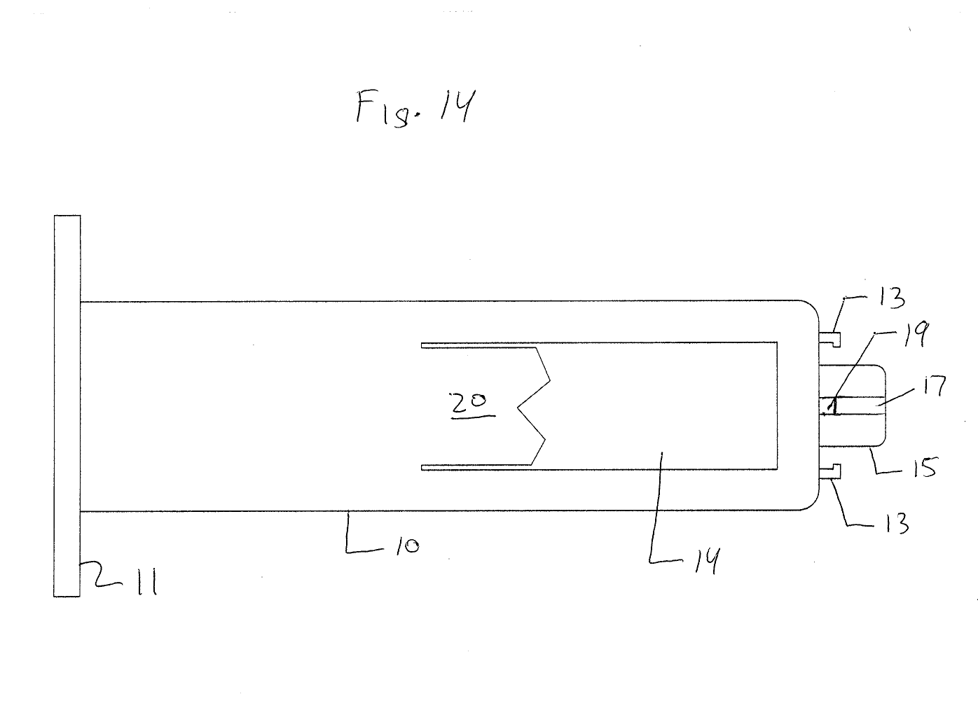

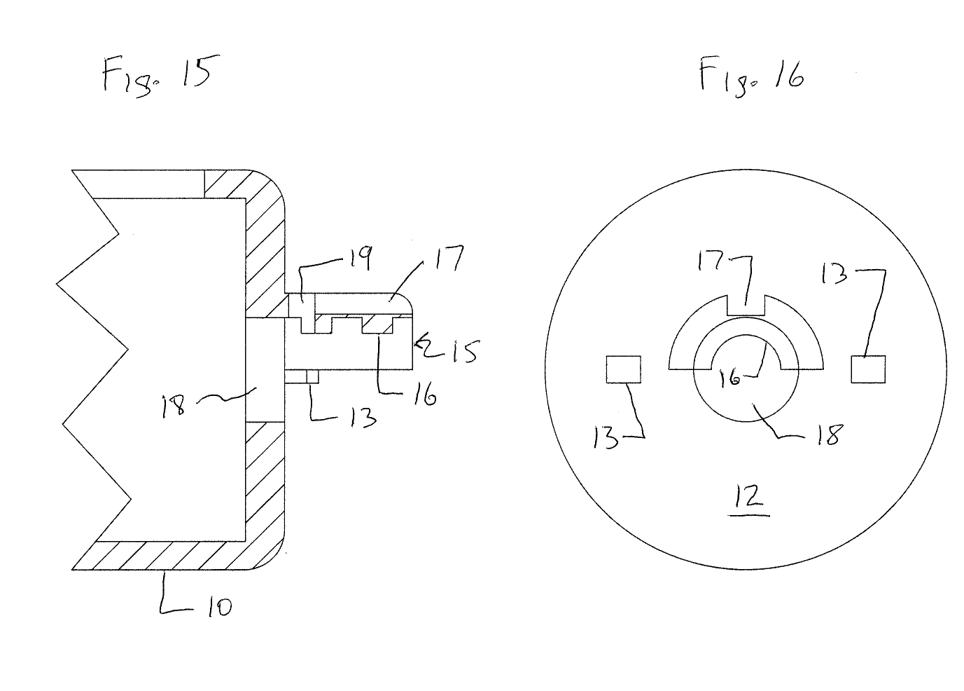

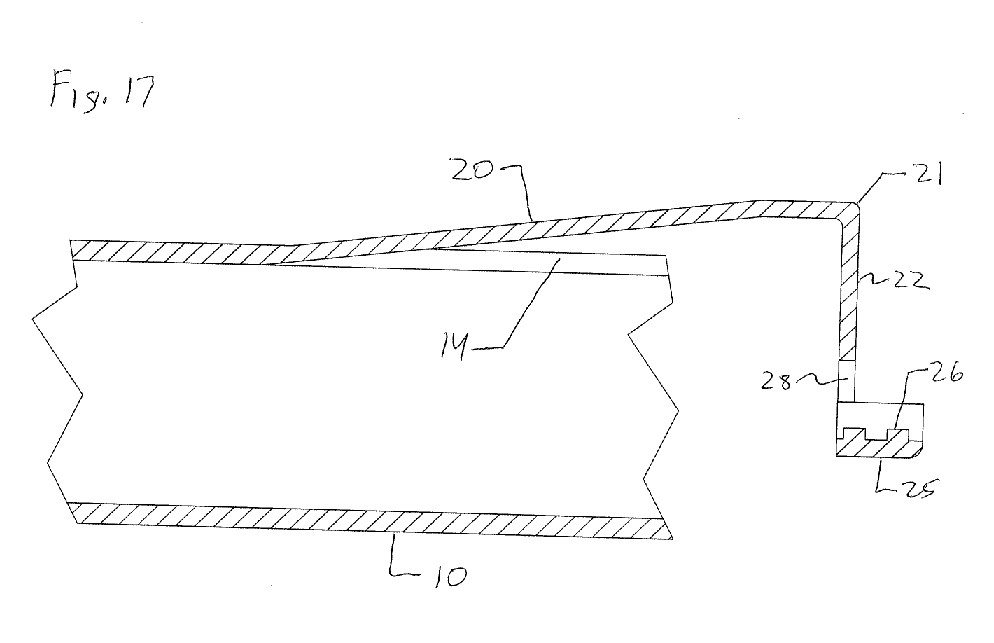

Referring now to the drawings and first to FIGS. 1-17 which shows a first non-limiting embodiment of a device 1 for injection and/or fluid collection according to the invention. The device 1 includes a generally cylindrical sleeve or body member 10 which includes a proximal end configured to allow an external needle 44 of a double-ended needle member or needle holder 40 to pass there through, and a distal end which can receive a collection container such as a vacutainer-type vial. In the embodiment of FIGS. 1-17, the distal end is open. However, it may also be closed off by a cap similar to the device disclosed in U.S. 2008/0262421 (U.S. Ser. No. 11/738,240) to SCHRAGA, the entire disclosure of which is hereby expressly incorporated by reference in its entirety. An external flange 11 is arranged at the distal end.

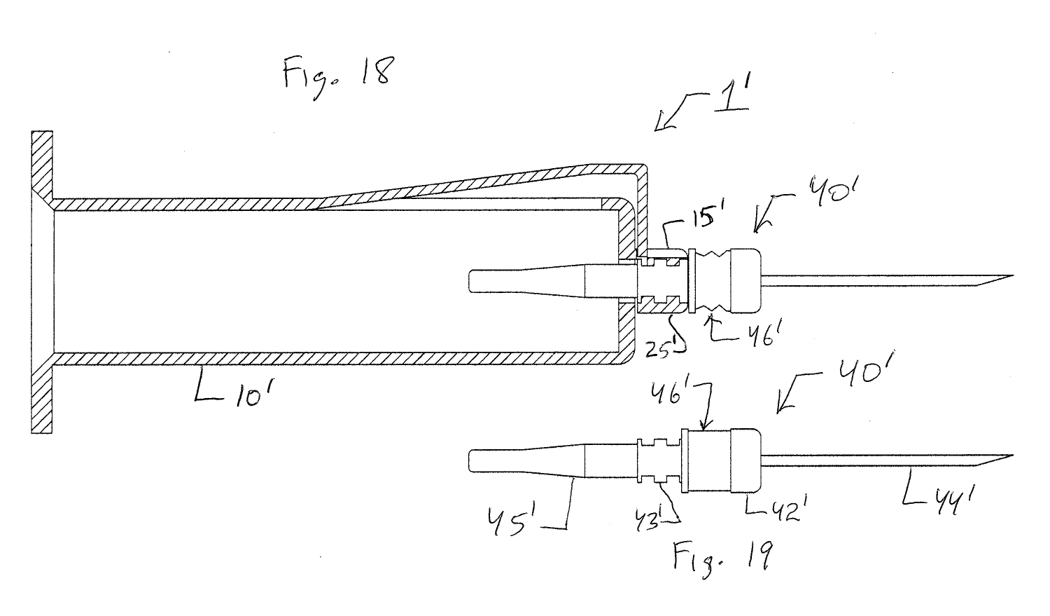

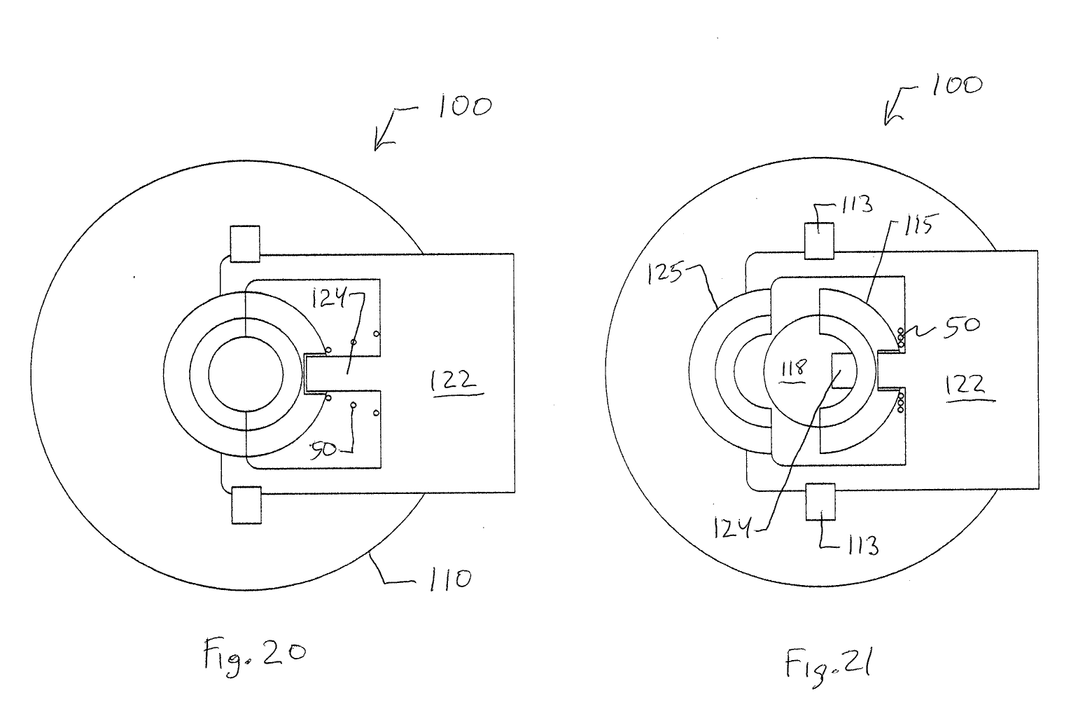

With reference to FIGS. 2 and 3, the needle holder or member 40 can be of any conventional type which can used with the type of device shown in FIG. 1. In embodiments, the member 40 includes a proximal needle 44, a distal needle 41, a retractable/protective cover member 45 and a hub section 42 from which the two needles 41 and 44 extend in opposite directions. The hub section 42 is sized and configured, e.g., has external thread-type feature, which can frictionally engage with inner comparable feature (which is described in detail below) so as to ensure that the member 40 is securely and axially retained when installed on the device (see FIG. 1).

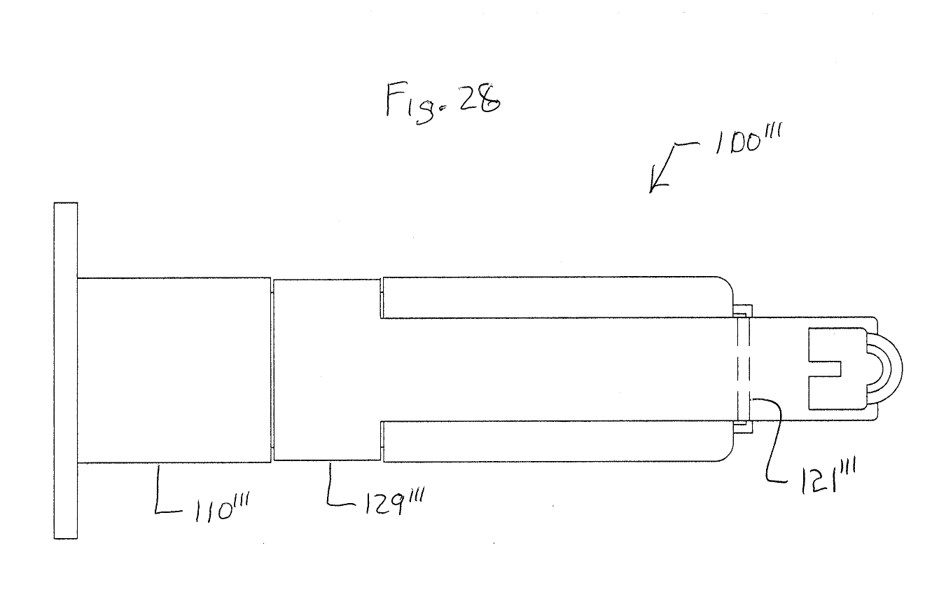

The device has a needle hub securing section arranged on the front or proximal end thereof. This section is structured and arranged to receive therein the needle member 40. The needle hub securing section comprises a fixed part 15 and a movable part 25. In embodiments, the fixed part 15 is integrally formed with the front end of the body 10. The movable part 25 is arranged on a free end of a member 20. In embodiments, the movable part 25 is integrally formed with the member 20. In embodiments, the other end of the member 20 is fixed to a portion of the main hollow section or body 10. In other embodiments, the other end of the member 20 is connected to a portion of the main hollow section 10 via a living hinge section LH. In embodiments, the other end of the member 20 is removably connected to a portion of the main hollow section 10 (see e.g., embodiment of FIGS. 30 and 31). In embodiments, the other end of the member 20 is integrally formed with the main hollow section 10 as is utilized in the embodiment of FIGS. 1-17. The deflectable member 20 includes a movable connecting section 22. In embodiments, the section 22 is fixed to a portion of the member 20. In other embodiments, the section 22 is connected to a portion of the member 20 via a living hinge 21. As is apparent from a comparison of FIGS. 5 and FIGS. 6 and 7, the section 22 can be bent over approximately 90 degrees and fillet into and between to retaining guides 13 arranged on a front end of the body 10. A projection 24 is arranged on or coupled to the section 22 (see FIG. 7). As is apparent from FIGS. 9 and 11, the projection 24 is configured to extend into an opening 19 (see FIG. 14) formed in the fixed part 15. The purpose of the projection 24 is to cause ejection of the member 40 when the deflectable member 20 is moved from an original position shown in FIGS. 6-9 to the ejection position shown in FIGS. 10-13.

With reference to FIGS. 6-9, it can be seen that the natural elasticity of the member 20 ensures that the section 22 is in an original position. As a result, the movable part 25, which is coupled thereto, is in a closed position by virtue of being in contact with the fixed part 15. These parts 15 and 25 are essentially semi-circular and when placed in contact with one another form an opening comprised of two semi-circular engaging sections 16 and 26. It is these sections 16 and 26 which frictionally engage with the engagement section 43 of the member 40. In order to install the member 40, so as to place the device 1 in the usable configuration of FIG. 1, the user applies a force F (see FIGS. 10-13) to the member 20 which causes the movable part 25 to move away from the closed position to the open position. This movement is a linear movement as a result of the sliding engagement between portions 27 and 28 of the section 22 and retaining guides 13. The retaining guides 13 have an upper or proximal shoulder (see FIG. 14) which ensures that section 22 (and specifically sections 27 and 28) maintain the bent configuration and prevent them from moving back to the position shown in FIGS. 4 and 5. At this point, a user can install the member 40 onto the body 10 by inserting the end 45 into the enlarged space formed between parts 15 and 25 until the annular surface of the section 42 contacts an annular proximal surface of the parts 15 and 25. The user can then remove the force F and allow the movable part 25 to again assume the position shown in FIGS. 6-9, which results in the device assuming the configuration of FIG. 1.

With reference to FIGS. 6-17, it can be seen that the body 10 has an opening 14 which allows for deflection movement of the member 20 noted above. An annular surface 12 defines a proximal end of the space formed in the section 10. A centrally disposed opening 18 allows the section 45 of the member 40 to pass into the space. As should be apparent from FIGS. 9, 11 and 13, in order to bend the section 22 90 degrees without damaging the projection 24, a slot or recess 17 is formed in the fixed part 15. To retain the section 22 in the bent configuration, the retainer guides 13 trap sections 27 and 28 there between.

The operation of the device of FIGS. 1-17 will now be described. According to one non-limiting embodiment, the device shown in FIGS. 4 and 5 is packaged. Once the package is open, a user bends section 22 along living hinge 21 and slightly deflects sections 27 and 28 until the section 22 is arranged between retaining guides 13 resulting in the configuration shown in FIGS. 6 and 7. Then, the user installs the needle member 40 so that the device 1 assumes the configuration of FIG. 1. This is accomplished when the user applies a force F (see FIGS. 10-13) to the member 20 which causes the movable part 25 to move away from the closed position to the open position. At this point, the user can install the member 40 onto the body 10 by inserting the end 45 into the enlarged space formed between parts 15 and 25 and into opening 18, and until the annular surface of the section 42 contacts an annular proximal surface of the parts 15 and 25. The user can then remove the force F and allow the movable part 25 to again assume the position shown in FIGS. 6-9, which results in the device assuming the configuration of FIG. 1. The user can then remove a safety cover (not shown) from the proximal needle 44, inject the same, e.g., into a human or animal, and then, e.g., insert a vacutainer-type vial into the space formed in the body 10. Once the device 1 is utilized to obtain a sample of desired amount and after the sample vial is removed from the interior space of the body 10, and after the injection needle 44 is removed from the injection site, the user can again apply the force F and move the movable part 25 to the open position shown in FIGS. 10-13. The mere opening of the space between parts 15 and 25 should allow the member 40 to drop out of the device 1. However, to prevent any sticking and/or to ensure that the member 40 is completely ejected from the device 1, the projection 24 engages with the section 43 to break any residual engagement stickiness between section 43 and section 16. The used needle member 40 can be safely discarded as a sharp. The device 1 can be reused or, preferably, discarded. Since the device is, in embodiments, essentially a one-piece member, its cost of manufacture is very low. This makes the cost incentive of re-use relatively low. As a result, the risks associated with reusing the device 1 with a new needle member 40 are eliminated and/or reduced. It is submitted that the cheaper the device 1 is to procure, the more likely that the user will use it only once and then discard it.

According to one non-limiting embodiment, the device shown in FIGS. 6 and 7 is packaged. Once the package is open, the user installs the needle member 40 so that the device 1 assumes the configuration of FIG. 1. Again, the is accomplished when the user applies a force F (see FIGS. 10-13) to the member 20 which causes the movable part 25 to move away from the closed position to the open position. At this point, the user can install the member 40 onto the body 10 by inserting the end 45 into the enlarged space formed between parts 15 and 25 and into opening 18, and until the annular surface of the section 42 contacts an annular proximal surface of the parts 15 and 25. The user can then remove the force F and allow the movable part 25 to again assume the position shown in FIGS. 6-9, which results in the device assuming the configuration of FIG. 1. The user can then remove a safety cover (not shown) from the proximal needle 44, inject the same, e.g., into a human or animal, and then, e.g., insert a vacutainer-type vial into the space formed in the body 10. Once the device 1 is utilized to obtain a sample of desired amount and after the sample vial is removed from the interior space of the body 10, and after the injection needle 44 is removed from the injection site, the user can again apply the force F and move the movable part 25 to the open position shown in FIGS. 10-13 and cause ejection of the member 40 as described above.

Referring now FIGS. 18 and 19, which show a second non-limiting embodiment of a device 1′ for injection and/or fluid collection according to the invention. The device 1′ includes a generally cylindrical sleeve or body member 10′ which is substantially similar to that of FIG. 1. However, this embodiment utilizes a differently configured double-ended needle member or needle holder 40′. In embodiments, the member 40′ includes a proximal needle 44′, a distal needle (not shown), a retractable/protective cover member 45′ and a hub section 42′ from which the two needles extend in opposite directions. The hub section 42′ is sized and configured, e.g., has external thread-type feature, which can frictionally engage with inner comparable feature, e.g., devices 16 and 26, so as to ensure that the member 40′ is securely and axially retained when installed on the device (see FIG. 18). Unlike the previous embodiment, the member 40′ also includes an elastic or biasing section 46′ which is compressed axially (i.e., moved to a retracted position) when the member 40′ is installed as shown in FIG. 18. This occurs because section 46′ is axially compressible and/or axially retractable. In embodiments, the section 46′ comprises an outer synthetic resin sleeve enclosing a spring. In embodiments, the section 46′ comprises a rubber-type member or sleeve which is elastically compressible axially. The advantage of this feature relates to the fact that when the user causes the movable part 25′ to move to the open position (in a manner similar to that shown in FIG. 10), the inherent biasing action of the section 46′ will automatically cause it to move back to an original or extended position (see FIG. 19). This, in turn, allows the member 40′ to release and eject quickly from engagement with the parts 15′ and 25′.

FIGS. 20 and 21 show a third non-limiting embodiment of a device 100 for injection and/or fluid collection according to the invention. This device 100 is similar to that shown in FIG. 1 (with comparable reference numbers increased by 100), except that it additionally utilizes a spring 50 to assist in biasing the section 122 (and therefore also section 125) towards the closed or original position. When the user causes the section 122 to move to the open position shown in FIG. 21, the spring 50 is compressed. As a result, when the user removes the force used to move section 122, the section 122 automatically moves to the closed position shown in FIG. 20 by the biasing force of the spring 50.

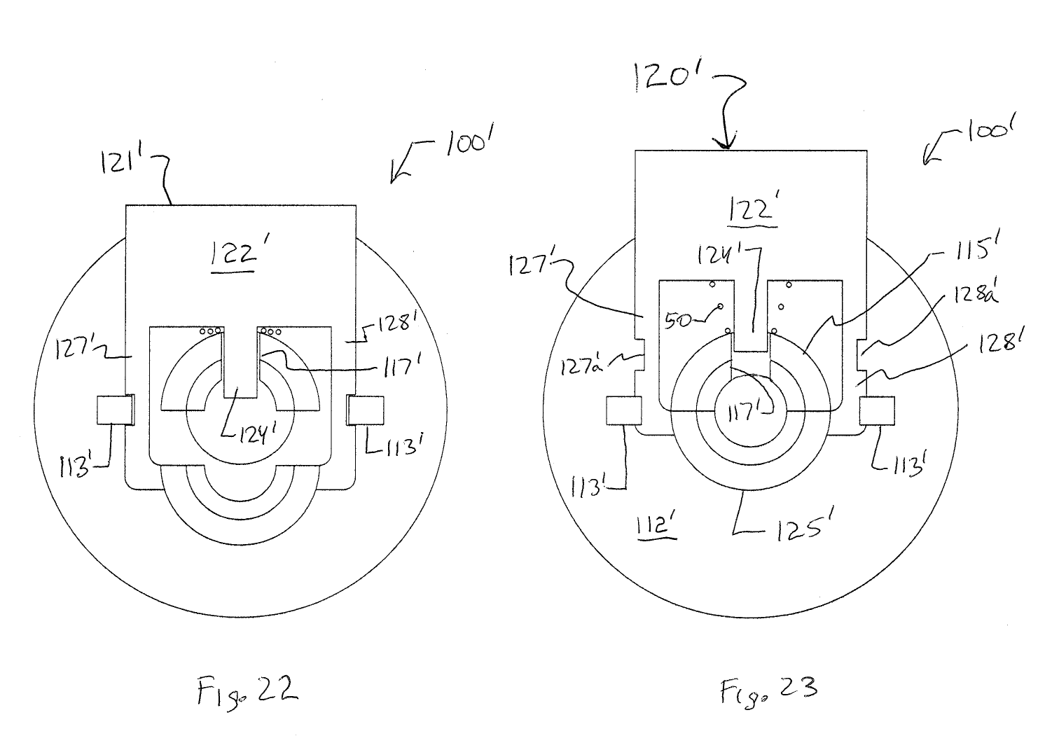

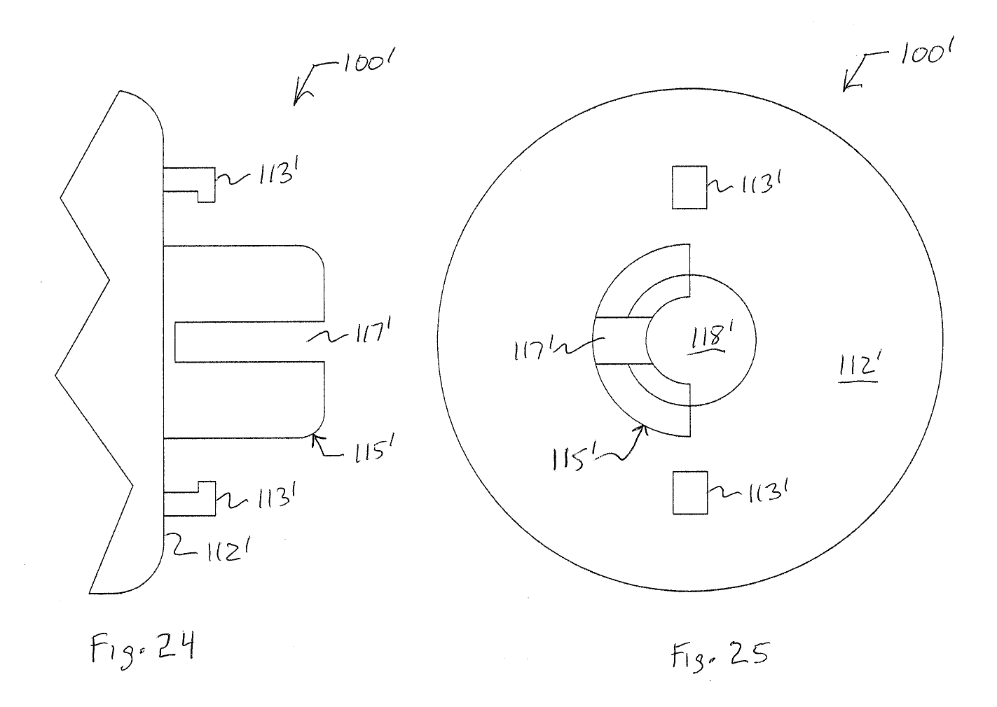

FIGS. 22-25 show a fourth non-limiting embodiment of a device 100′ for injection and/or fluid collection according to the invention. This device 100′ is similar to that shown in FIGS. 20 and 21, except that it utilizes an open slot 117′ configuration which makes it easier to receive therein the projection 124′ as well as side slots 127′a and 128′a to make it easier to fully install and/or insert the section 122′ in between the retaining guides 113′. As is apparent in FIG. 22, to assembly the section 122′ properly, a user can bend the section 122′ about 90 degrees, align the side slots 127′a and 128′a with the retaining guides 113′, and then push the section 122′ into and between the retaining guides 113′. Once the user releases his/her grip of the section 122′, the deflectable member 120′ (owing to its natural elasticity as well as the biasing force provided by the spring 50), will move and cause the section 122′ to move to the position shown in FIG. 23. In embodiments, the location of the side slots 127′a and 128′a relative to that of the retaining guides 113′ is such that once the user moves the member 120′ to the open position shown in FIG. 22 (such as would occur after use), the section 122′ can pivot and/or deflect outwards along the living hinge 121′ so that the section 122′ no longer engages with the retaining guides 113′. When the section 122′ disengages from the retaining guides 113′, this both renders the device 100′ un-usable (unless the user attempts to re-assembly the section 122′ between the retaining guides 113′) and provides a visual indication to the user (and others) that the device 100′ has already been used and should be discarded. In embodiments, the location of the side slots 127′a and 128′a relative to that of the retaining guides 113′ is such that once the user moves the member 120′ to the open position shown in FIG. 22 (such as would occur after use), the side slots 127′a and 128′a are not exactly aligned with the retaining guides 113′. As a result, the section 122′ cannot pivot and/or deflect outwards along the living hinge 121′. Instead, the section 122′ continues to engage with the retaining guides 113′. However, this alignment can be such that upon using more force (or another force applied at another location such as directly against part 125′) than is typically used to move part 125′ to the open position, the section 122′ disengages from the retaining guides 113′. In the this embodiment, this larger force can be used only to assemble the section 122′ between the retaining guides 113′.

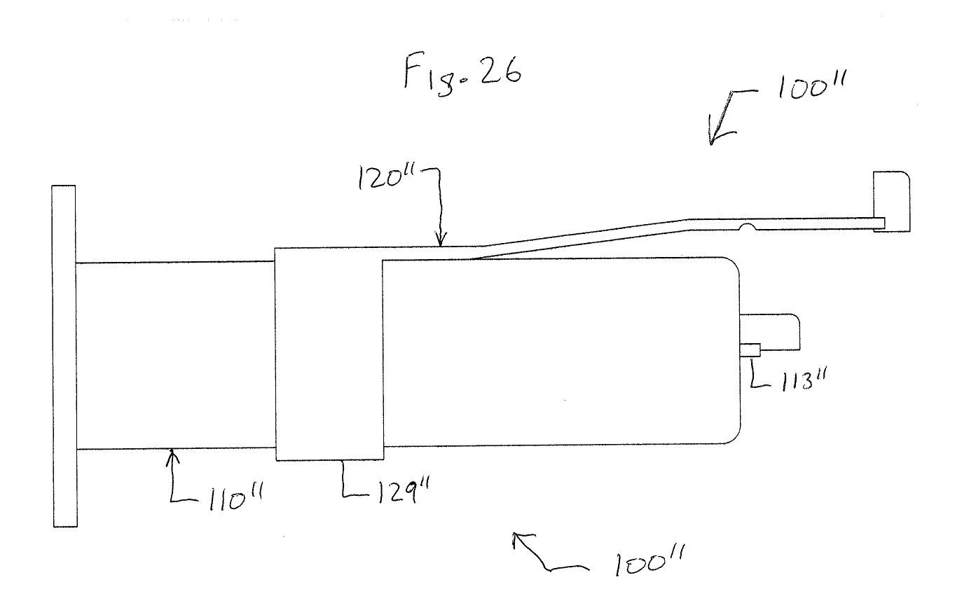

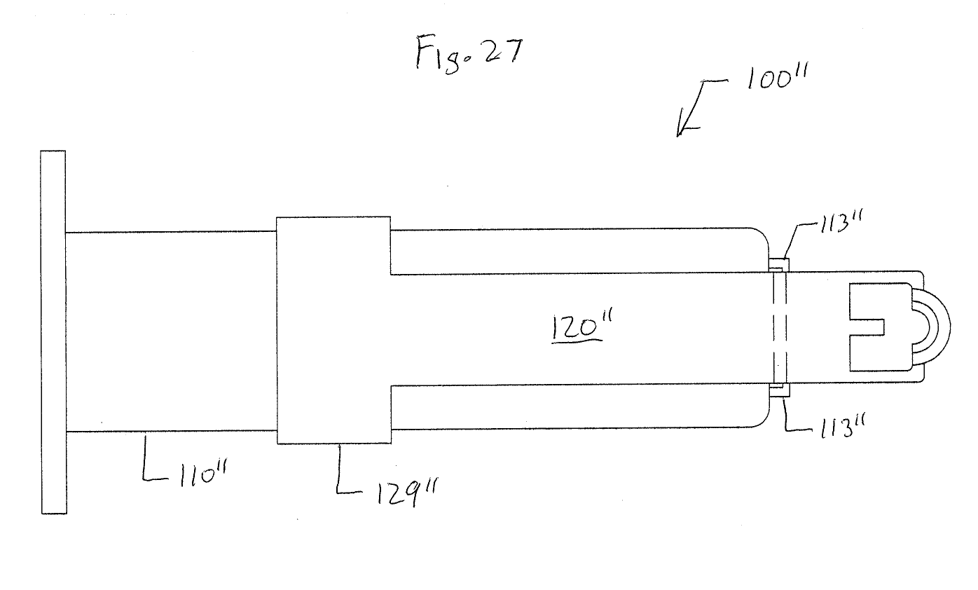

FIGS. 26 and 27 show a fifth non-limiting embodiment of a device 100″ for injection and/or fluid collection according to the invention. This device 100″ is similar to that shown in FIG. 1, except that the member 120″ is not integrally formed with the body 110″. In embodiments, the member 120″ is formed separately from the body 110″ and thereafter secured to the body 110′ via section 129″. In embodiments, the member 120″ is removable secured to the body 110″.

FIGS. 28-31 show a sixth non-limiting embodiment of a device 100′″ for injection and/or fluid collection according to the invention. This device 100′″ is similar to that shown in FIGS. 26 and 27, except that the section 129′″ is sized and configured to seat within a generally circumferential recess 130′″ of the body 120′″. An advantage of this arrangement is that it ensures the proper location of the living hinge 121′″, which, in turn, facilitates assembly. To ensure that the member 120′″ is easily installed and/or removed, the section 129′″ is generally C-shaped and provides for clip-on attachment of the member 120′″.

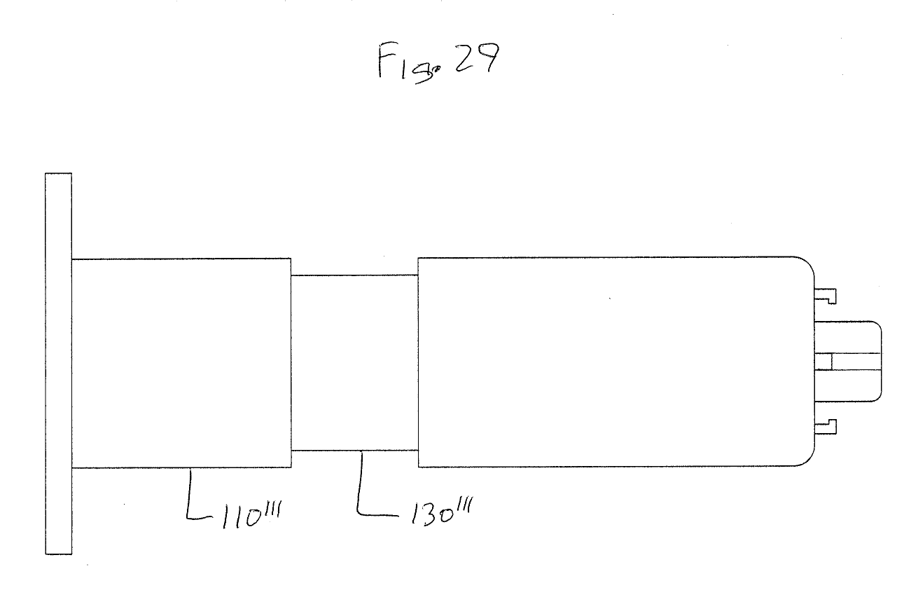

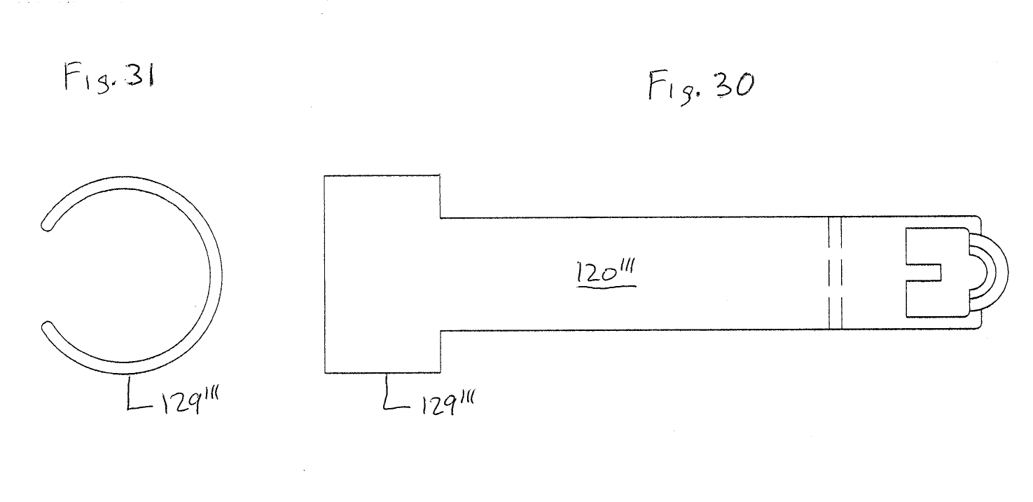

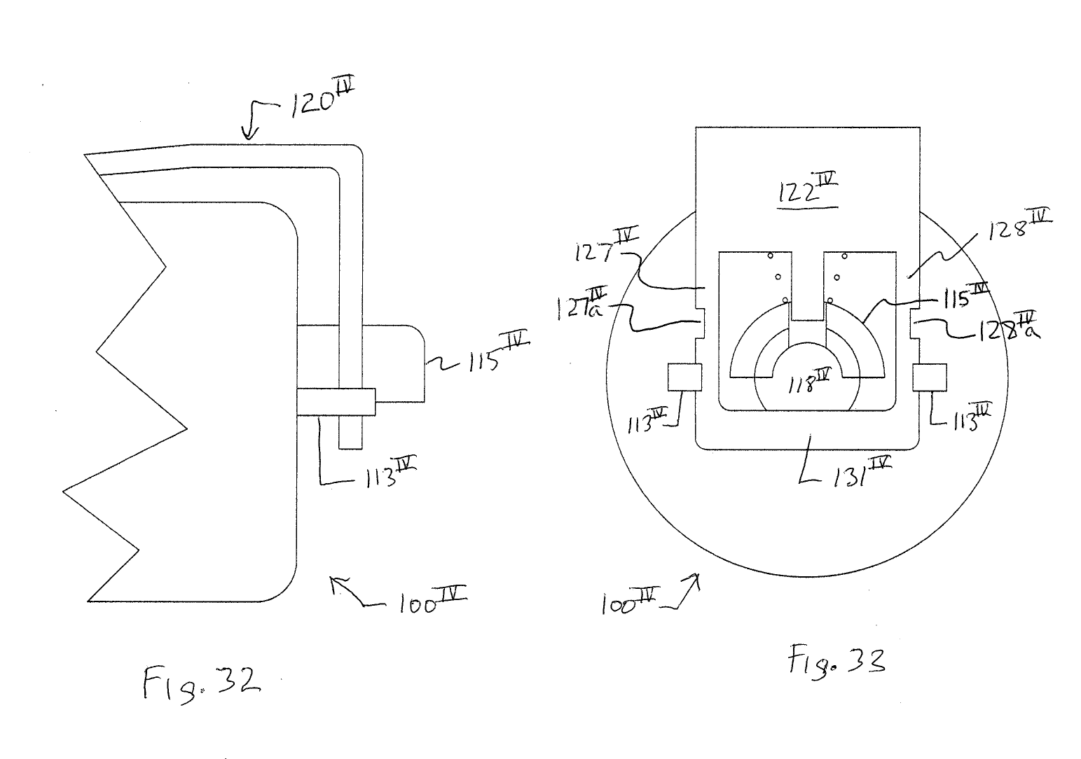

FIGS. 32-35 show a seventh non-limiting embodiment of a device 100^{IV} for injection and/or fluid collection according to the invention. This device 100^{IV} is similar to that shown in FIGS. 22 and 23, except that the movable part (e.g., member 125′ in FIGS. 22 and 23) is not utilized. Instead, a cross-member 131^{IV} is utilized to trap the needle member 40 against the fixed part 115^{IV} in the closed position. In embodiments, the cross-member 131^{IV} is integrally formed with the section 122^{IV}.

The device 100^{IV} of FIGS. 32-35 utilizes side slots 127^{IV}a and 128^{IV}a to make it easier to fully install and/or insert the section 122^{IV} in between the retaining guides 113^{IV}. As should be apparent from FIGS. 34 and 35, to assembly the section 122^{IV} properly, a user can bend the section 122^{IV} about 90 degrees, align the side slots 127^{IV}a and 128^{IV}a with the retaining guides 113^{IV} (using slightly more force than is applied in FIGS. 34 and 35) and then push the section 122^{IV} into and between the retaining guides 113^{IV}. Once the user releases his/her grip of the section 122^{IV}, the deflectable member 120^{IV} (owing to its natural elasticity as well as the biasing force provided by the spring 50), will move and cause the section 122^{IV} to move to the position shown in FIGS. 32 and 33. In embodiments, the location of the side slots 127^{IV}a and 128^{IV}a relative to that of the retaining guides 113^{IV} is such that once the user moves the member 120^{IV} to the open position shown in FIGS. 34 and 35 (such as would occur after use), the side slots 127^{IV}a and 128^{IV}a are not exactly aligned with the retaining guides 113^{IV}. As a result, the section 122^{IV} cannot pivot and/or deflect outwards along the living hinge 121^{IV}. Instead, the section 122^{IV} continues to engage with the retaining guides 113^{IV}. However, this alignment can be such that upon using more force (or another force applied at another location such as directly against part 131^{IV}) than is typically used to move part 131^{IV} to the open position, the section 122^{IV} disengages from the retaining guides 113^{IV}. In the this embodiment, this larger force can and/or need be used only to assemble the section 122^{IV} between the retaining guides 113^{IV}.

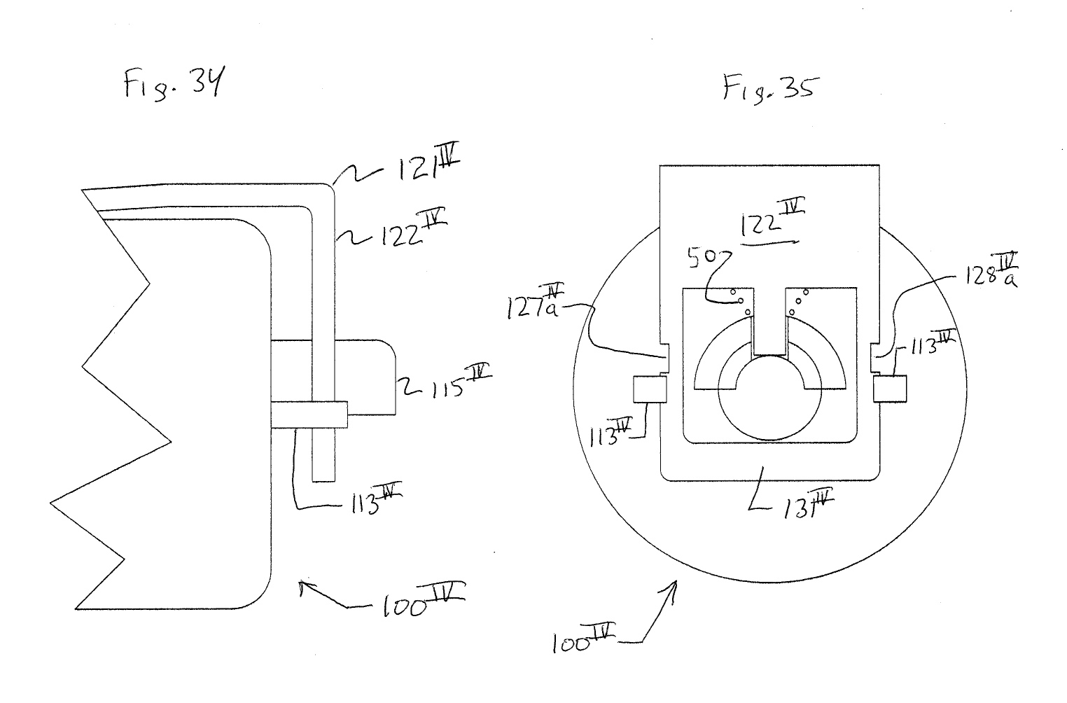

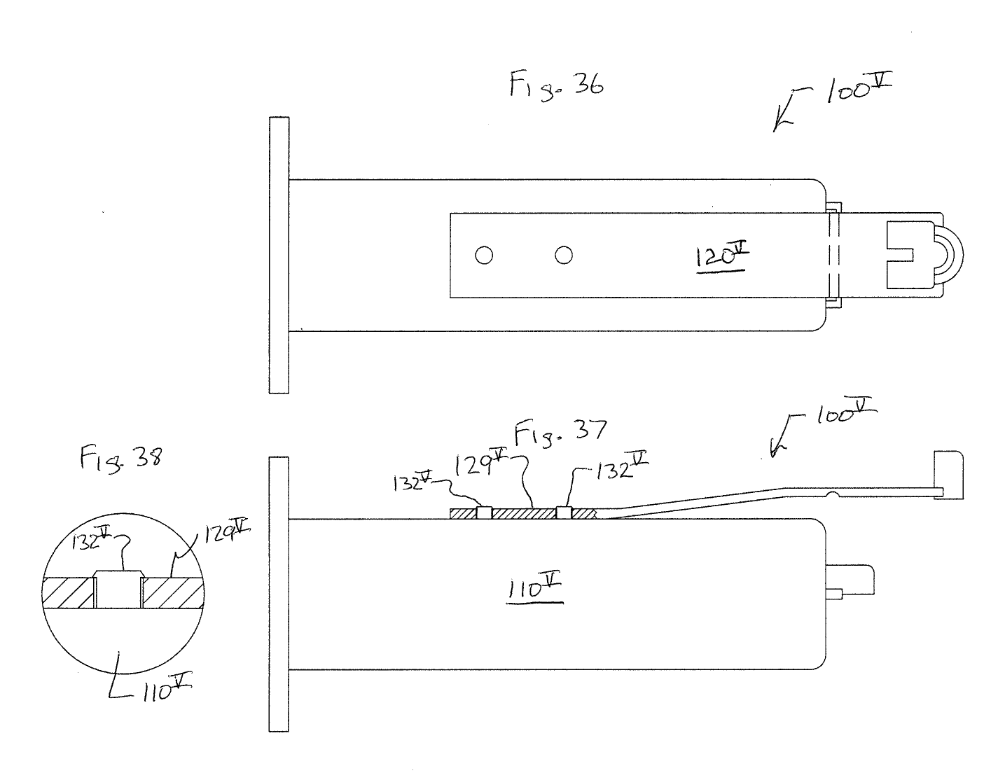

FIGS. 36-38 show an eighth non-limiting embodiment of a device 100^{V} for injection and/or fluid collection according to the invention. This device 100^{V} is similar to that shown in FIG. 1, except that the member 120^{V} is not integrally formed with the body 110^{V}. In embodiments, the member 120^{V} is formed separately from the body 110^{V} and thereafter secured to the body 110^{V} via section 129^{V}. In embodiments, the member 120^{V} is non-removable secured to the body 110^{V} via projections and/or rivets 132^{V} integrally formed with the body 110^{V}. The rivets 132^{V} extend into openings formed in the member 120^{V}. A secure attachment occurs when the rivets 132^{V} have their free ends deformed or otherwise enlarged to prevent removal of the member 120^{V}. In embodiments, other securing arrangements can also be utilized such as ultrasonic welding, adhesive bonding, etc,.

FIGS. 39 and 40 show a ninth non-limiting embodiment of a device 100^{VI} for injection and/or fluid collection according to the invention. This device 100^{VI} is similar to that shown in FIG. 1, except that the body 110^{VI} is not a one-piece integrally formed member. Instead, the rear flange 111^{VI} of the body of the fluid sampling device 100^{VI} is a separately formed member that can be secured to the main portion of the body via, e.g., engagement between a circumferential projection 133^{VI} and a circumferential recess 111^{VI}b. The flange 111^{VI} includes a generally circumferential section 111^{VI}a which extends over a rear or distal portion of the main portion of the body 101^{VI}. In embodiments, the flange 111^{VI} can rotate relative to the body 10^{VI}, while being axially retained thereto via the projection 133^{VI} and recess 111^{VI}b. This rear flange arrangement can be utilized on any of the herein disclosed embodiments.

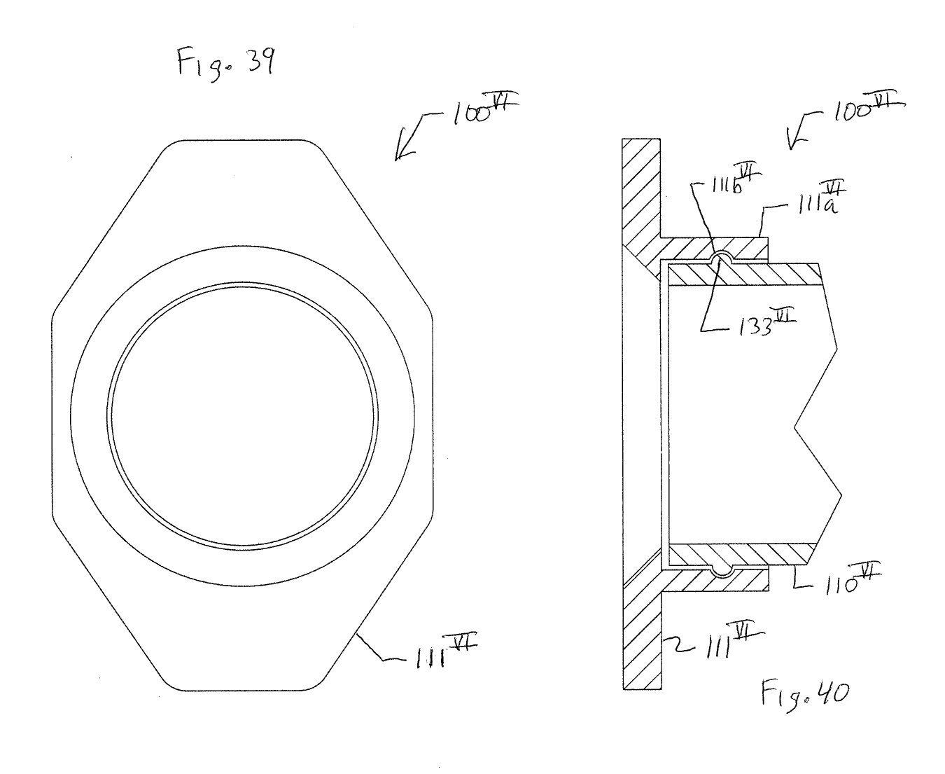

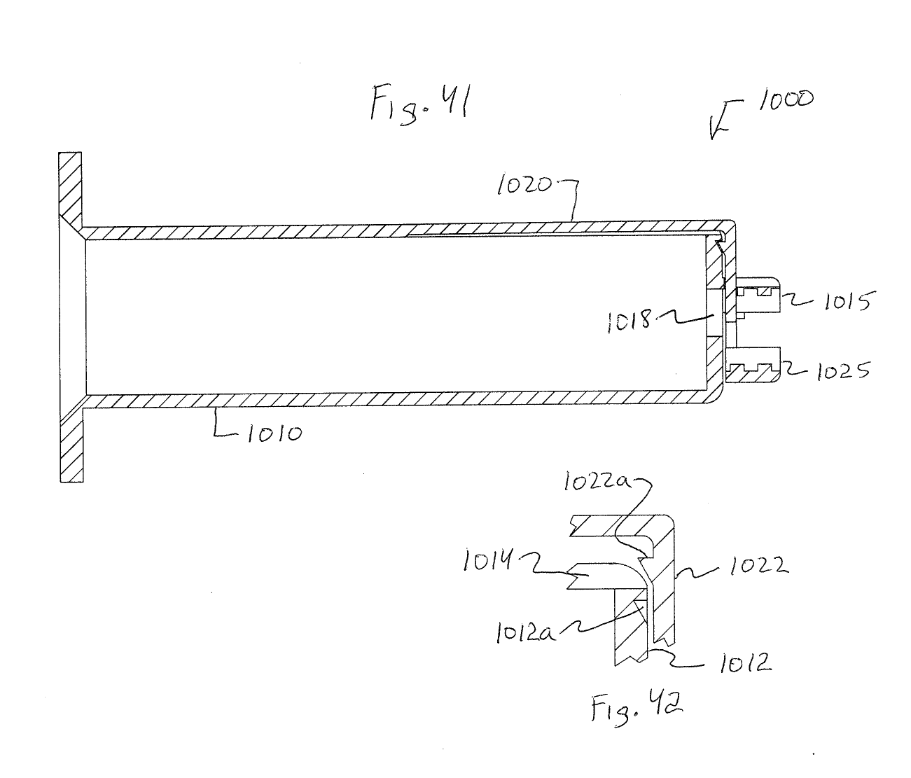

FIGS. 41 and 42 show a tenth non-limiting embodiment of a device 1000 for injection and/or fluid collection according to the invention. This device 1000 is similar to that shown in FIG. 1 (with comparable reference numbers increased by 1000), except that it utilizes a system for locking it in the open position to prevent re-use of the fluid sampling device 1000. In embodiments, the locking system includes one or more tapered projections 1022a arranged on the section 1022 of member 1020 and one or more tapered recesses 1012a arranged on and/or formed in the proximal wall 1012 of the body 1010.

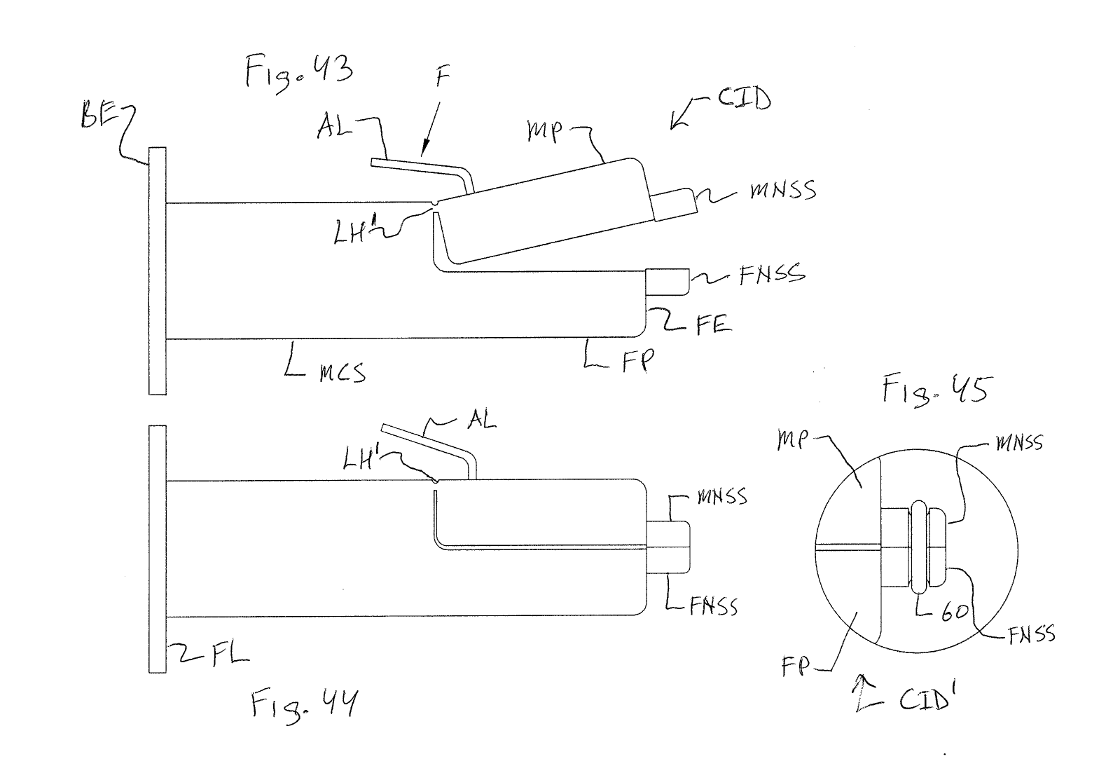

FIGS. 43 and 44 show an eleventh non-limiting embodiment of a device CID for injection and/or fluid collection according to the invention. This device CID is different from that shown in FIG. 1. The device CID has a needle hub securing section arranged on the front or proximal end FE. This section is structured and arranged to receive therein the needle member 40 (not shown in FIGS. 44 and 44). The needle hub securing section comprises a fixed needle member support part FNSS and a movable needle support part MNSS. In embodiments, the part FNSS is integrally formed with the fixed portion FP of the body and is arranged on a front end FE of the body or main cylindrical section MCS. The movable part MNSS is arranged on a free end of a member MP. In embodiments, the movable part MNSS is integrally formed with the member MP. In embodiments, the other end of the member MP is fixed to a portion of the main hollow section or body MCS. In other embodiments, the other end of the member MP is connected to a portion of the main hollow section MCS via a living hinge section LH′. In embodiments, the other end of the member MP is integrally formed with the main hollow section MCS as is utilized in the embodiment of FIGS. 43 and 44. The deflectable member MP includes the movable part MNSS. In embodiments, an actuating lever AL is fixed to and/or integrally formed with a portion of the member MP in order to allow a user to place the device CID in the open position shown in FIG. 43 by applying a force F thereto. As in previous embodiments, the body MCS includes a rear flange FL arranged at a back end BE thereof. In embodiments, the natural elasticity of the living hinge LH′ ensures that the member MP returns to the closed position of FIG. 44 when the force F is removed.

FIG. 45 shows a twelfth non-limiting embodiment of a device CID′ for injection and/or fluid collection according to the invention. This device CID′ is similar to that of FIGS. 43 and 44. However, the device CID′ additionally includes an elastic ring 60 arranged in a circumferential recess formed in the parts MNSS and FNSS. The ring 60 functions to bias the movable part MNSS towards the closed position shown in FIG. 45.

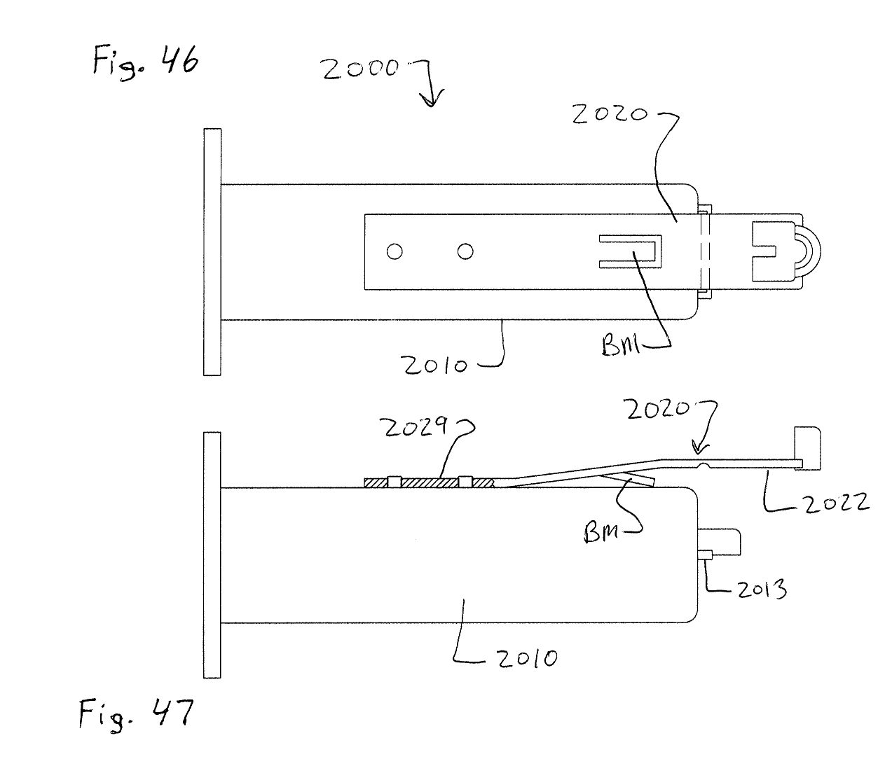

FIGS. 46 and 47 show a thirteenth non-limiting embodiment of a device 2000 for injection and/or fluid collection according to the invention. This device 2000 is similar to that shown in FIG. 1, except that the member 2020 is not integrally formed with the body 2010. In embodiments, the member 2020 is formed separately from the body 2010 and thereafter secured to the body 2010 via section 2029. In embodiments, the member 2020 is non-removable secured to the body 2010 via projections and/or rivets integrally formed with the body 2010. The rivets extend into openings formed in the member 2020. A secure attachment occurs when the rivets have their free ends deformed or otherwise enlarged to prevent removal of the member 2020. In embodiments, other securing arrangements can also be utilized such as ultrasonic welding, adhesive bonding, etc,. To ensure that the member 2020 is biased towards the closed position, the member 2020 includes a biasing member BM. In embodiments, the natural elasticity of the biasing member BM ensures that the member 2020 returns to the closed position (after the section 2022 is bent over and retained in members 2013). In embodiments, the biasing member BM is integrally formed with the member 2020.

The devices described herein can also utilize one or more features disclosed in the prior art documents expressly incorporated by reference herein. Furthermore, one or more of the various parts of the device can preferably be made as one-piece structures by e.g., injection molding, when doing so reduces costs of manufacture. Non-limiting materials for most of the parts include synthetic resins such as those approved for syringes, blood collection devices, or other medical devices. Furthermore, the invention also contemplates that any or all disclosed features of one embodiment may be used on other disclosed embodiments, to the extent such modifications function for their intended purpose.

It is noted that the foregoing examples have been provided merely for the purpose of explanation and are in no way to be construed as limiting of the present invention. While the present invention has been described with reference to an exemplary embodiment, it is understood that the words which have been used herein are words of description and illustration, rather than words of limitation. Changes may be made, within the purview of the appended claims, as presently stated and as amended, without departing from the scope and spirit of the present invention in its aspects. Although the present invention has been described herein with reference to particular means, materials and embodiments, the present invention is not intended to be limited to the particulars disclosed herein; rather, the present invention extends to all functionally equivalent structures, methods and uses, such as are within the scope of the appended claims.

What is claimed:

1. A fluid collection/injection device comprising:
a body having a front end, a back end, and a main hollow section arranged between the front and back ends;
a needle hub securing section arranged on the front end and being structured and arranged to receive therein a needle member; and
the needle hub securing section comprises a fixed part and a movable part,
wherein the fixed part is integrally formed with the front end and the movable part is arranged on a member that has one end which is one of:
fixed to a portion of the main hollow section;
connected to a portion of the main hollow section via a living hinge;
removably connected to a portion of the main hollow section; and
integrally formed with the main hollow section.

2. The device of claim 1, wherein the body is one of generally cylindrical and generally tubular.

3. The device of claim 1, wherein the fixed part and the movable part form a generally cylindrical section which the movable part is in an initial position.

4. The device of claim 1, wherein the fixed part and the movable part each generally comprise one-half of an internal locking thread structured and arranged to engage with an external thread of the needle member.

5. The device of claim 1, wherein the member has one end which is fixed to the portion of the main hollow section.

6. The device of claim 1, wherein the member has one end which is removably connected to the portion of the main hollow section.

7. The device of claim 1, wherein the member has one end which is integrally formed with the main hollow section.

8. The device of claim 1, wherein the member has one end which is connected to the portion of the main hollow section via the living hinge.

9. The device of claim 1, wherein the body and the needle hub securing section comprise a one-piece member.

10. The device of claim 1, wherein the device is a one-piece member.

11. The device of claim 1, further comprising guide projections arranged on the front end of the body.

12. The device of claim 1, wherein the member comprises a projection for facilitating removal of the needle member.

13. The device of claim 1, wherein the member comprises an opening sized to receive therein the fixed part.

14. The device of claim 1, wherein the member comprises a finger engaging section which is arranged on a side of the fixed part opposite the movable part.

15. The device of claim 1, wherein the member comprises a finger engaging section which is movable towards the body when it experiences a force and which moves back to an initial position away from the body when the force is removed.

16. The device of claim 1, wherein the member comprises a finger engaging section which is movable towards the body when it experiences a force and which automatically moves back to an initial position away from the body when the force is removed.

17. The device of claim 1, wherein the front end comprises a through opening sized to receive an inner needle end of the needle member.

18. The device of claim 1, wherein the rear end comprises a flange.

19. The device of claim 1, wherein the rear end comprises a rotatable and/or movably mounted flange.

20. The device of claim 1, further comprising a mechanism for locking the movable part in a position which prevents the removable needle member from being reinstalled or locked to the body.

21. The device of claim 1, further comprising a mechanism for preventing re-use of the device.

22. The device of claim 1, further comprising a mechanism for rendering the device single-use.

23. The device of claim 1, further comprising a biasing mechanism biasing the movable part towards the fixed part.

24. A method of taking a fluid sample using the device of claim 1, the method comprising:
installing the needle member onto the device;
inserting a receptacle into the device; and
removing the receptacle from the device.

25. A method of taking a fluid sample using the device of claim 1, the method comprising:
inserting a receptacle into the device; and
removing the receptacle from the device.

26. A method of taking a fluid sample using the device of claim 1, the method comprising:
inserting a receptacle into the device;
removing the receptacle from the device; and
preventing re-use of the device.

27. A sampling or injecting device comprising:
a generally cylindrical body having a front end, a back end, and a main hollow section arranged between the front and back ends;
a needle hub securing section arranged on the front end and being structured and arranged to receive therein a removable needle member; and
the needle hub securing section comprises a fixed part and a movable part which together function to secure the removable needle member to the body,
wherein the fixed part is integrally formed with the front end and the movable part is arranged on a member that has one end which is one of:
fixed to a portion of the main hollow section;
connected to a portion of the main hollow section via a living hinge;
removably connected to a portion of the main hollow section; and
integrally formed with the main hollow section.

28. A blood sampling device comprising:
a body having a front end, a back end, and a main hollow section arranged between the front and back ends;
a needle hub securing section arranged on the front end and being structured and arranged to receive therein a needle member; and
the needle hub securing section comprises a fixed part and a movable part which together function to secure the removable needle member to the body,
wherein the fixed part is integrally formed with the front end and the movable part is arranged on a member that has one end which is one of:
fixed to a portion of the main hollow section;
connected to a portion of the main hollow section via a living hinge;
removably connected to a portion of the main hollow section; and
integrally formed with the main hollow section.
